(19) United States
(12) Patent Application Publication
(10) Pub. No.: US 2015/0132369 A1
(43) Pub. Date: May 14, 2015

(54) NANOLIPOSOMAL CYCLOSORIN FORMULATIONS FOR IMMUNOSUPPRESION AND METHODS FOR THE PRODUCTION THEREOF

(71) Applicant: Exir Nano Sina Company, Tehran (IR)

(72) Inventors: S. Mahdi Rezayat, Tehran (IR); Alireza Partoazar, Tahran (IR); Kambiz Gilani, Tehran (IR)

(73) Assignee: Exir Nano Sina Company, Tehran (IR)

(21) Appl. No.: 14/536,667

(22) Filed: Nov. 9, 2014

Related U.S. Application Data
(60) Provisional application No. 61/902,155, filed on Nov. 9, 2013.

(51) Int. Cl. A61K 9/127; A61K 47/24; A61K 45/06; A61K 47/28; A61K 38/13

(52) U.S. Cl. CPC A61K 9/127; A61K 47/28; A61K 38/13; A61K 45/06; A61K 47/24
USPC 424/450; 514/20.5

(57) ABSTRACT

A liposomal formulation for targeting immunotherapy, with synergistic effect in the case of encapsulating an immunosuppressive drug, such as Cyclosporine A, and the method of preparing the same. The reduced toxicity and significant reduction of Delayed-Type Hypersensitivity DTH) due to extremely reduced dosage, potential therapeutic value in control of chronic transplant rejection, allergies and certain autoimmune diseases, and increased efficacy are some of numerous and significant benefits of the present invention.

### CROSS REFERENCE TO RELATED APPLICATION

The present invention claims priority from pending U.S. Provisional Patent Application Ser. No. 61/902,155, filed Nov. 9, 2013, entitled “Highly Potent Nanoliposomal Cyclosporin: A Formulation for Immunosuppression,” the subject matter of which is incorporated by reference herein in its entirety.

### SPONSORSHIP STATEMENT

This application has been sponsored by the Iranian Nanotechnology Initiative Council, which does not have any rights in this application.

### TECHNICAL FIELD

This application relates to the fields of biochemistry and medicine, and in particular to an improved liposomal formulation. In addition, the instant application relates to a liposomal formulation for immunotherapy purposes, which could have synergistic effects when entrapping an immunosuppressive drug, such as cyclosporine A, and a method of preparation of this composition.

### BACKGROUND OF THE INVENTION

Cyclosporine, formerly known as cyclosporine A (hereinafter “CsA”), marketed under the brand name Sandimmune®, is a high lipophilic cyclic polypeptide immunosuppressant agent having 11 amino acids with a solubility of 9 μg/mL in water. CsA is produced as a metabolite by the fungus species Beauveria nivea. CsA is a powerful immunosuppressive drug, which is used to prevent rejection of transplanted organs, and it is also used for the treatment of several autoimmune and some parasitic diseases, as well as in multiple sclerosis, asthma and allergies.

CsA causes suppression of delayed-type hypersensitivity reactions and selectively inhibits T cell activation and proliferation. Based on the CsA mechanism of action, using CsA in a variety of other T cell-mediated diseases has been reported, e.g. in the treatment of Behçet's acute ocular syndrome, endogenous uveitis, atopic dermatitis, inflammatory bowel disease, and nephritic syndrome.

However, there are some adverse reactions to cyclosporine therapy, such as renal dysfunction, severe toxicities to the kidney, liver and central nervous system, which are limitations for long-term administration of CsA. Other common side effects of cyclosporine include headache, dizziness, paresthesia, neuropathy, tremor, hypertension, hyperlipidemia, nephropathy, acne, hirsutism, and gum hyperplasia.

In addition, Cremophor, a polyoxyethylated castor oil, one of the commercial vehicles for CsA in intravenous formulation, is associated with acute renal failure and may lead to anaphylactic shock. As is known from the prior art, its toxicity is dose- and time-dependent. However, constant intravenous infusions have been associated with a lesser degree of renal dysfunction.

As is known to those of skill in the pertinent art, parenteral and oral administrations of lipophilic drugs are difficult due to their low water solubility.

Phospholipids have amphipathic characteristics and can be used to solubilize particular drugs. While the phospholipid bilayers are diffused in water, lipid vesicles containing a lipophilic zone are created between acyl chains and hydrophilic aqueous compartment in core, spontaneously. Therefore, they can encapsulate hydrophilic drugs, along with binding to lipophilic and amphipathic drugs.

After introducing intravenous liposomal formulations, various lipophilic drugs, such as Amphothericin B (Albelcet®, AmBisome®) and benzoporphyrin (Visudyne®, Verteporfin for injection) were developed and successfully introduced to the market. In the case of CsA, the prior art shows the effects of its liposomal composition in the reduction of toxicity and increasing the efficacy of CsA in in-vitro and in-vivo assays.

Locally delivering immunosuppressive agents to a transplanted organ can be considered as an option for avoiding the complications of systemically delivered immunosuppressants. As is known from the prior art, the delivery of immunosuppressive agents toward a transplanted liver, leads to increased efficacy, as well as decreased adverse effects, such as nephrotoxicity. Furthermore, lymphatic targeting tissue and cells could be considered as an important strategy in the reduction of other organ's toxicities. Accumulation of liposomes in lymph nodes and the spleen is usually administrated by intraperitoneal (IP) injection. Results of previous studies have shown great accumulation of stealth-pH sensitive liposomes in spleen (with major lymphatic system), than nonfusogenic and nonpegylated liposomal formulations.

In addition, fusogenic liposomal CsA demonstrated significantly greater inhibition on T cell proliferation than other positively or negatively charged liposomes. In fact, the delivery of encapsulated macromolecules, such as peptides, proteins and DNA into the cytosol is the unique characteristic of fusogenic liposomes. Delivery of the encapsulated contents into the cell cytoplasm leads to maximizing drug availability to the sites of action in the cells. However, it should be understood in the art that CsA is a peptide and easily degrades in the lysosome by lysosomal enzymes.

DOPE (DioleoylPhosphoethanolamine) has an important role in accumulation of drugs in lymphatic cells by endosomal escape of CsA and availability of the entire drug to cytosol. During endocytosis, internalized cell membrane vesicles containing liposomes fuse with endosomes having an internal acidic pH (around 5.0) in which, the bilayer assembly of DioleoylPhosphoethanolamine (DOPE) changes to a hexagonal structure and facilitates the transfer of the liposome contents (in the bilayer as well as the inner compartment) to cytosol before endosome fuses with lysosome.

Furthermore, cholesterol causes elevation of CsA entrapment in liposomes and increases the stability of liposomes in plasma. Since liposomes are accompanied by phosphatidylserine, immunosuppressive phospholipid formulation shows synergistic response by CsA to the reduction of Delayed-Type Hypersensitivity (DTH).

Liposomal formulations having phosphatidyl serine (PS) have both anti-inflammatory and immunosuppressive effects. Therefore, using phosphatidylserine (PS) in the formulation, such as disclosed in the present invention, explains the synergistic effects. The anti-inflammatory effects of phosphatidylserine liposomes resemble those of dexamethasone that could reduce the delayed phase of carrageenan-triggered mouse paw edema. Phosphatidylserine (PS) has also resulted in T cell inhibition activity, as well as a reduction in the number of total leukocytes in immunized mice.

It should be understood that the invention could be used as well for the delivery of different kinds of immune suppressants, such as Tacrolimus (also FK-506 or fujimycin, or with trade names of Prograf, Advagraf, Protopic), as is understood in the art.

The liposomal compositions disclosed herein have several benefits as a drug delivery system for CsA in suppression of the immune system based on the following reasons: (1) targeting the lymphatic cells and organs, which results not only in toxicity reduction of the vital organs, but also ensures an increase in immunosuppressive activity; (2) liposomal compositions containing phosphatidylserine result in powerful immunosuppressive effects, beside using low doses of the drug which leads to reduced toxicity or adverse effects of CsA; and (3) the large capacity of present liposomal formulations for entrapment of hydrophobic agents in the phospholipid bilayer, its low toxicity, easy preparation method, controlled size distribution and reproducibility are some of the many benefits of the present formulation.

There is, accordingly, a present need to provide improved compositions for use in drug delivery.

It is, therefore, an object of the present invention to provide improved compositions and methods of manufacture that produce beneficial nanoliposomal compositions for said drug delivery, such as in situations involving immunosuppression activity.

It is also an object of the present invention to reduce Delayed-Type Hypersensitivity and other ailments by having a reduced dosage required.

These objects are met in various embodiments of the present invention where there is a synergistic effect in the usage of the compositions of the present invention, such as when encapsulating Cyclosporine A and other compounds. As a result of this advancement in the technology, there is not only a reduction in the required dosage, and consequent reduced toxicity, the compositions of the present invention have therapeutic value in transplant rejections, allergies, autoimmune diseases and other situations. Accordingly, the improved, nanoliposomal compositions, and method therefor of the present invention offer significant advantages over the known prior art.

### SUMMARY OF THE INVENTION

In one embodiment, the present invention is directed to a nanoliposomal cyclosporin A composition having high efficiency in the inhibition of Delayed-Type Hypersensitivity (DTH) reactions, details for which are presented as set forth and described further hereinbelow.

Another embodiment of the present invention is directed to the synergistic effects of the present liposomal composition, with therapeutic applications in immunosuppression therapy due to the combination of cyclosporine A in said liposomal composition, which also comprises DioleoylPhosphoethanolamine (DOPE), Dipalmitoylphosphatidylcholine (DPPC), Dipalmitoylphosphatidylserine (DPPS), Cholesterol (Chol), Distearoylphosphoethanolamine-polyethyleneglycol-2000 (DSPE-PEG 2000) and other components and admixtures. The presence of the aforementioned components in the formulation leads to the entrapment of liposomes in a size range of about 150 nm in the spleen.

In another embodiment, the present liposomal formulation is an easy and reproducible method having the following exemplary steps: (1) mixing phospholipids with cyclosporine A; (2) dissolving the resultant mixture in chloroform; (3) removing the solvent by a rotary evaporator which results in the deposition of a thin lipid film on the flask wall; (4) hydrating the lipid film in a buffer; (5) rotating the container for a few minutes for separating a lipid film containing vesicles in the top layer of the suspension; (6) preparing mono-dispersed nanoliposomes in a range of 150 nm using polycarbonate membranes; and (7) storing the resultant suspension at 4° C.

In still another embodiment of the present invention, the inventive liposomal composition was used in an in vivo Cell-Mediated Immune response model, which is a classical delayed-type hypersensitivity reaction in mice able to determine T cell proliferation and activation responses to antigens and active agents. The immunosuppressive effects were observed in a single dose of liposomal CsA (2.5 mg/kg) in comparison with the control group, as well as intravenous (IV) cyclosporine A (CsA) formulation.

In a further embodiment of the present invention, the suppression of DTH, in addition to considerable reduction of immunosuppressant dosage, was observed, which resulted in lower organ toxicities and extended usage of medications, particularly in severe and chronic autoimmune diseases and transplantations.

### BRIEF DESCRIPTION OF THE DRAWINGS

While the specification concludes with claims particularly pointing out and distinctly claiming the subject matter that is regarded as forming the present invention, it is believed that the invention will be better understood from the following description taken in conjunction with the accompanying DRAWINGS, where like reference numerals designate like structural and other elements, in which:

FIG. 1 illustrates the effects of using different formulations of an embodiment of the present invention 12, 24 and 36 hours after sensitization on Delayed-Type Hypersensitivity (DTH) responses to Ship Red Blood Cell (SRBC), which indicates greater immunosuppressive activity (P<0.05) of liposome-CsA formulations 24 hours after the sensitization;

FIG. 2 illustrates the effects of different formulations of an embodiment of the present invention 24 hours after sensitization in mice spleen mass, in which significantly lower spleen mass numbers were observed for liposome-CsA (P<0.05) compositions, liposomal compositions and a negative control group than with a positive control group;

FIG. 3 illustrates the effects of various formulations of an embodiment of the present invention 24 hours after sensitization on average size of the germinal centers of mice, which indicates lower size average of GCs (p<0.01) in spleen sections for a liposome-CsA group compared to a positive control group (p<0.01), pursuant to the teachings and principles of the present invention; and

FIG. 4 illustrates histograms of the germinal centers of mice affected by various compounds 24 hours after sensitization, pursuant to the teachings of the present invention.

### DETAILED DESCRIPTION OF THE INVENTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

It should be understood by a person skilled in the art that the present invention is directed to and designed for systemic immunosuppression with significantly low dose regimens of immunosuppressant in a synergistic manner, in which invented nanoliposomes are characterized by long circulation in blood, targeting of lymphatic cells and organs, overcoming lysosomal degradation and having immunosuppressive activity, compositions and methods of preparation for which are described in more detail hereinbelow.

The material and substances used for preparing the liposomal compositions used in the instant invention include: DioleoylPhosphoethanolamine (DOPE), Dipalmitoylphosphatidylcholine (DPPC), Dipalmitoylphosphatidylserine (DPPS), Cholestrol (Chol), Distearoylphosphoethanolamine-polyethyleneglycol-(DSPE-PEG 2000), and mixtures thereof, as is understood in the art. The presence of DOPE, DPPS, DSPE-PEG 2000 and other components together in the composition increase the entrapment of liposomes, in sizes of about 150 nm in the spleen, a major lymphatic system.

In other embodiment of the present invention, the liposomal compositions have a synergist effect by entrapping an immunosuppressive drug, such as cyclosporine A. It should be understood that the best and most efficient ratio of the lipid content in a liposomal composition delimited as: DOPE/DPPC/DPPS/Chol/DSPE-PEG 2000 is 30:35:10:25:5, respectively. Targeting lymphatic organs, especially the spleen, is achieved by long circulating fusogenic nanoliposomes in a size range of about 100-150 nm in diameter.

### Example 1

### Preparation of Liposomes

As discussed hereinabove, the lipid content in the formulation in one embodiment of the present invention is comprised of DioleoylPhosphoethanolamine (DOPE), Dipalmitoylphosphatidylcholine (DPPC), Cholesterol (Chol), Dipalmitoyl phosphatidylserine (DPPS), Distearoylphosphoethanolamine-polyethyleneglycol-2000 (DSPE-PEG 2000) in a ratio of (DOPE/DPPC/DPPS/Chol/DSPE-PEG 2000:30:35:10:20:5, respectively), while, 70 mg lipid and 3 mg Cyclosporine defined as liposome-CsA.

To prepare the liposomal composition, 70 mg lipid content is mixed with 10 cyclosporine A in the presence of chloroform at 37° C. in a round-bottom flask. After completely dissolving, the solvent is removed under backpressure by a rotary evaporator within 6 hours. A phosphate buffer (pH 7.4) is added to a final drug concentration of 0.3 mg/ml, and the lipid film is hydrated for 15 minutes. Then, vesicles are dispersed, while rotating in the presence of small glass beads at 45° C. for 15 minutes, and the lipid film is separated completely. The suspension should then be kept at room temperature for about 1 hour.

To prepare mono dispersed nanoliposomes in a range of 150 nm in diameter, first, the equipment of an extruder containing a liposome suspension is heated at 59° C. on a hot plate. Then, the suspension is extruded repeatedly by 1000, 400 and 100 nm polycarbonate membranes, at least 3 times, to prepare a uniform nano-sized liposomal suspension with liposomal diameters of about 150 nm. Then, the suspension is stored at about 4° C.

The particles' size distribution and the zeta potential of liposomes were determined by a Zetasizer, as is understood in the art.

### Determination of Encapsulation Efficiency

To determine the encapsulation efficiency of a modified formula pursuant to the teaching of the present invention, first, the consumed polycarbonate filters are immersed in methanol, then the filtrated liposomal solution are filtrated again with 30 KD Amicon tube (Centrifugal Filter Device) by centrifugation at 6000 rpm at 4° C., which is defined as ultrafiltration. The amount of CsA after each step filtration is measured by a UV spectrophotometer at 214 nm. Then, the encapsulation efficiency of CsA in liposomes was calculated using the following formula.

$$\% Encapsulation efficiency = 100 \times \frac{( \begin{matrix}total amount CsA added - \\ amount CsA recovered in filters and ultrafiltration\end{matrix} )}{total amount CsA added}$$

With reference to TABLE 1 herein below, there is shown in detail, dosage and characteristics of different formulations, such as may be prepared pursuant to embodiments of the present invention and the teachings thereof.

TABLE 1

| | | Dosage | Characteristics |
|---|---|---|---|
| Compound | Formulation | (mg/kg) | (approximately) |
| Vehicle | Castrol oil (Crel) | — | — |
| Cyclosporine A | CsA-Crel | 2.5, 60 | — |
| (i.v | | | |
| formulation) | | | |
| liposome-CsA | DOPE/DPPC/DPPS/ | 70 lipid | Zeta potential: −30 |
| | Chol/DSPE-PEG2000 & | 2.5 CsA | Size: 120-150 nm |
| | CsA | | in diameter |
| | | | Encapsulation |
| | | | efficiency(%): >80% |

### Example 2

### Classical Delayed-Type Hypersensitivity Reaction Test in Mice

To investigate T cells activation and suppression in response to antigens and suppressants, the Delayed-Type Hypersensitivity (DTH) response test in mice was applied. Responses were observed in the region of challenging antigens of Ship Red Blood Cell (SRBC) that immunized mouse earlier with the same antigen. The amount of responses was assessed by measuring the increase in the thickness of mice footpads. The maximum reaction is usually observed 24 hours after sensitization.

### Hypersensitivity Induction and Animal Treatment

At least six male inbreed mice aged 8-12 weeks were selected randomly for each group. The aforementioned groups included the control (treated with Castrol oil), the liposomal composition (without admixing with drug), liposome Cyclosporine A (as 2.5 mg/kg CsA), cyclosporine A 2.5 and 6-mg/kg groups. Also, negative and positive control groups, which are defined as with and without hypersensitivity induction groups, respectively, were added to the group. Mice were immunized with s.c injection of 0.5 ml 108 washed SRBC (Ship Red Blood Cell) in saline and were challenged 4 days later under the left hind footpad with 0.05 ml with SRBC for providing sensitization.

Treatments are administrated in a single dose as i.p (intra peritoneal) as described hereinabove in TABLE 1, in which there are Liposome, Intravenous (IV), CsA formulations (2.5 and 60 mg/kg), liposomal CsA (2.5 mg/kg), and vehicle (i.v CsA formulation, CsA-Crel).

### Example 3

### Immunosuppressive Activity Measurement

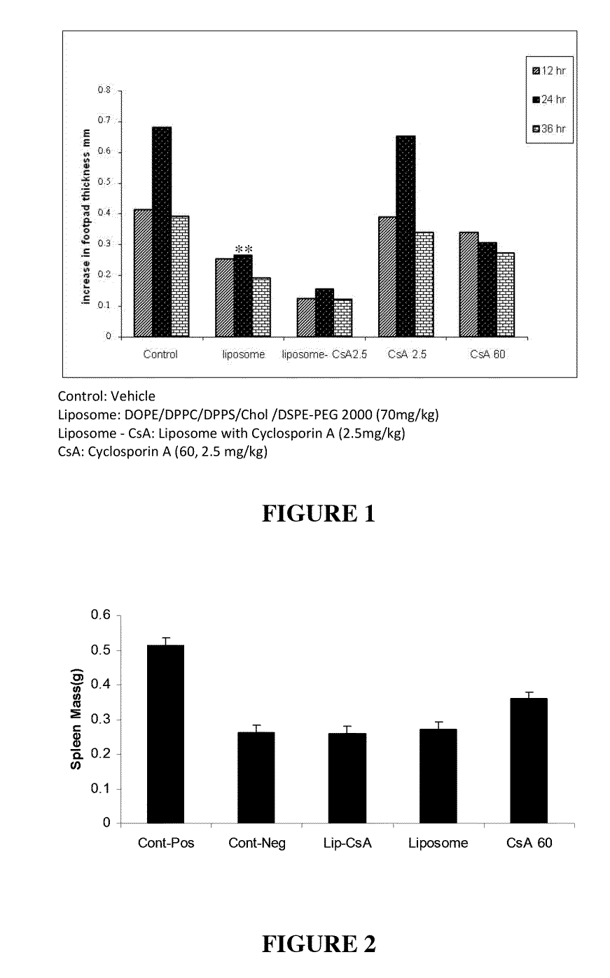

Edema was induced in the left hind paw of all the immunized mice. The paw thickness was measured from the ventral to the dorsal surfaces using a dial caliper before and 12, 24 and 36 hours after sensitization. The edema was calculated as the thickness variation before and after SRBC injection. The results are illustrated and described in more detail in the charts set forth and described in connection with FIG. 1 of the DRAWINGS.

Immunosuppressive activity is expressed the same as the inhibition percentage of edema when compared with the control group and was calculated using the following formula:

Inhibition %=(1−Tt/Tc)×100

where Tₜ and T_{c} are the thickness variation of the test group and the control group, respectively.

With reference now to TABLE 2 hereinbelow, there is shown in detail the results of immunosuppressive activity of compounds in mice.

TABLE 2

| | | Inhibition (%) | | |
|---|---|---|---|---|
| Compound | CsA Dose (mg/Kg) | at 12 h | at 24 h | at 36 h |
| Control(Vehicle) | — | 0 | 0 | 0 |
| Liposome | — | 39 | 61 | 51 |
| CsA | 60 | 18 | 55 | 30 |
| CsA | 2.5 | — | — | — |
| Liposome-CsA | 2.5 | 70% | 77% | 68% |

### Statistics

The various acquired data were analyzed statistically by one-way analysis of variance (ANOVA) followed by a Tukey multi-comparison test. Results with P<0.05 were considered to be statistically significant. As noted in TABLE 3 hereinbelow, the results of mortality percentage after administration of compounds in mice is observed.

TABLE 3

| Compound | Mortality % |
|---|---|
| liposome | — |
| Liposome-CsA (2.5 mg/kg) | — |
| Cyclosporin A (60 mg/kg) | 30 (approximately) |
| Vehicle (CsA-Cre) | — |

By highlighting and comparing the results illustrated in FIG. 1 and in TABLE 2 hereinabove, the immunosuppressive activity of liposome-CsA formulation (P<0.05) is significantly greater than the control group, mainly 24 hours after sensitization. In other words, the immunosuppressive activity of liposome-CsA formulation is greater and more effective than other compounds 12 hours, 24 hours and 36 hours after sensitization. The percent of DTH inhibition by liposome-CsA was approximately 70%, 77% and 68% in 12 hours, 24 hours and 36 hours, respectively, and for liposomes without CsA, this amount was approximately 39%, 61% and 51% in 12 hours, 24 hours and 36 hours, respectively.

The results also indicated that Immunosuppressive activities of liposome-CsA were greater than CsA 60 mg/kg free form in all of the evaluation times. Moreover, no mortality was observed several days after using high dose administrations of the liposome-CsA formulation in mice, while 30% mortality was observed during immunization by using cyclosprin A (60 mg/kg) free form, as set forth and illustrated in connection with TABLE 3 hereinabove.

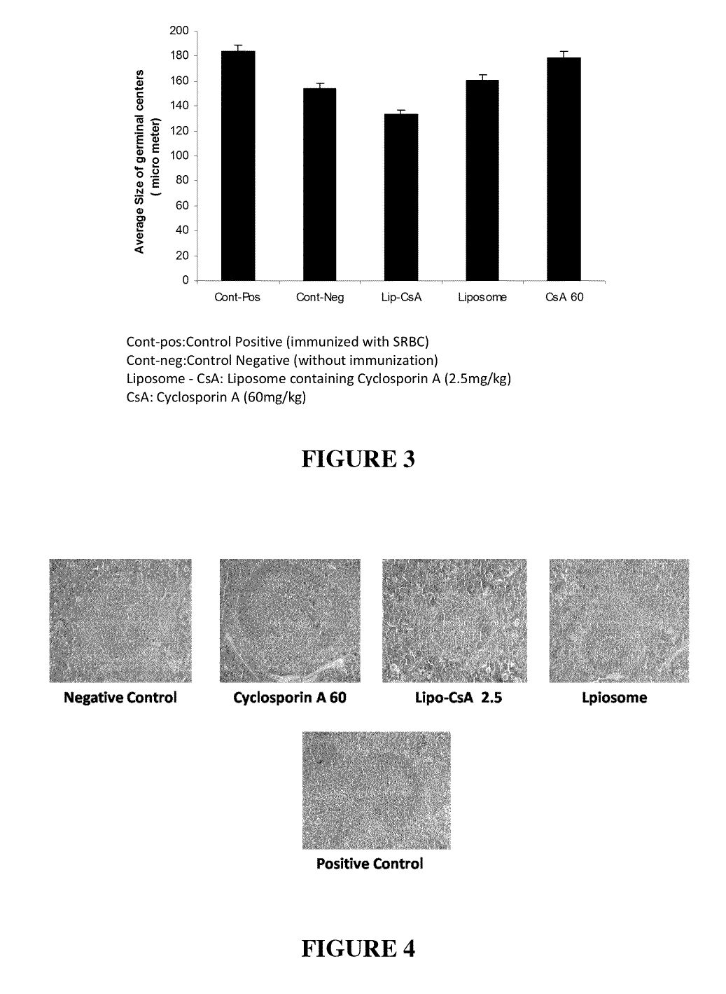

The presence of massive spleen and large germinal centers (GCs) in both positive control and CsA (60 mg/kg) groups, respectively, are illustrated in FIG. 2 and FIG. 3 of the DRAWINGS.

In contrast, paucity of spleen mass and GC formation in spleen of mice happened in liposome-CsA, liposome and negative control groups. Quantitative analysis revealed lower numbers for spleen mass (p<0.05) and average size of GCs (p<0.01) in spleen sections of liposome-CsA groups, as compared with the positive control groups.

As a result, this liposomal formulation is suggested for CsA delivery, as well as the other immunosuppressive drugs, for a potent immunosuppression therapy. The present invention, for example, could be used according to the mechanism of action of CsA for certain immunotherapy diseases, transplantation and inflammation in case of i.v and i.p administration. The formulation set forth in the instant invention brings considerable beneficial properties, such as targeting inflammation organs and grafts, as well as CsA toxicities reduction, because of the decreased dosage regimen.

While the present invention has been illustrated by the description of the embodiments thereof, and while the embodiments have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the breadth or scope of the applicant's concept. Furthermore, although the present invention has been described in connection with a number of exemplary embodiments and implementations, the present invention is not so limited but rather covers various modifications and equivalent arrangements, which fall within the purview of the appended claims.

What is claimed is:

1. A nanoliposomal composition comprising:
at least one lipid,
wherein said lipid comprises cholesterol and at least one phospholipid, and
wherein said nanoliposomal composition is used for immunotherapy.

2. The nanoliposomal composition according to claim 1, wherein said at least one phospholipid is selected from the group consisting of phosphatidylserine, phosphatidylethanolamine, and mixtures thereof.

3. The nanoliposomal composition according to claim 2, wherein the phosphatidylethanolamine is selected from the group consisting of DioleoylPhosphoethanolamine (DOPE), Distearoylphosphoethanolamine (DSPE), phosphatidylcholine, a Distearoylphosphoethanolamine conjugated with polyethylene glycol (PEG) to make Distearoylphosphoethanolamine-polyethylene glycol (DSPE-PEG 2000), and mixtures thereof.

4. The nanoliposomal composition according to claim 3, wherein said DioleoylPhosphoethanolamine (DOPE) is present in an amount from about 10 to about 50% of the total lipid content.

5. The nanoliposomal composition according to claim 4, wherein said DioleoylPhosphoethanolamine (DOPE) is present in an amount from about 20 to about 40% of the total lipid content.

6. The nanoliposomal composition according to claim 5, wherein said DioleoylPhosphoethanolamine (DOPE) is present in an amount from about 25 to about 35% of the total lipid content.

7. The nanoliposomal composition according to claim 3, wherein said Distearoylphosphoethanolamine-polyethylene glycol (DSPE-PEG 2000) is present in an amount from about 0.1 to 20% of the total lipid content

8. The nanoliposomal composition according to claim 7, wherein said Distearoylphosphoethanolamine-polyethylene glycol (DSPE-PEG 2000) is present in an amount from about 1% to about 10%.

9. The nanoliposomal composition according to claim 8, wherein said Distearoylphosphoethanolamine-polyethylene glycol (DSPE-PEG 2000) is present in an amount from about 4% to about 10% of the total lipid content.

10. The nanoliposomal composition according to claim 3, wherein said phosphatidylcholine is Dipalmitoylphosphatidylcholine (DPPC).

11. The nanoliposomal composition according to claim 10, wherein said Dipalmitoylphosphatidylcholine (DPPC) is present in an amount from about 10% to about 50% of the total lipid content.

12. The nanoliposomal composition according to claim 11, wherein said Dipalmitoylphosphatidylcholine (DPPC) is present in an amount from about 20% to about 40% of the total lipid content.

13. The nanoliposomal composition according to claim 12, wherein said Dipalmitoylphosphatidylcholine (DPPC) is present in an amount from about 30% to about 40% of the total lipid content.

14. The nanoliposomal composition according to claim 3, wherein a combination of said DioleoylPhosphoethanolamine (DOPE), dipalmitoylphosphatidylserines (DPPS), and DSPE-PEG 2000 together in said nanoliposomal composition increase the entrapment of liposomes therein in sizes ranging from about 50 nm to 150 nm in a targeted cell or organ.

15. The nanoliposomal composition according to claim 2, wherein the phosphatidylserine is dipalmitoylphosphatidylserines (DPPS).

16. The nanoliposomal composition according to claim 15, wherein said dipalmitoylphosphatidylserines (DPPS) is present in an amount from about 10% to about 50% of the total lipid content.

17. The nanoliposomal composition according to claim 16, wherein said dipalmitoylphosphatidylserines (DPPS) is present in an amount from about 20% to about is 40% of the total lipid content.

18. The nanoliposomal composition according to claim 17, wherein said dipalmitoylphosphatidylserines (DPPS) is present in an amount from about 30% to about 35% of the total lipid content.

19. The nanoliposomal composition according to claim 1, wherein said cholesterol is present in an amount from about 1 to about 50% of the total lipid content.

20. The nanoliposomal composition according to claim 19, wherein said cholesterol is present in an amount from about 10 to about 40% of the total lipid content.

21. The nanoliposomal composition according to claim 20, wherein said cholesterol is present in an amount from about 15 to about 25% of the total lipid content.

22. The nanoliposomal composition according to claim 1, wherein the ratio of DioleoylPhosphoethanolamine (DOPE)/Dipalmitoylphosphatidylcholine (DPPC)/dipalmitoylphosphatidylserines (DPPS)/Cholesterol/Distearoylphosphoethanolamine-polyethylene glycol (DSPE-PEG 2000) is about 30:35:10:25:5.

23. A nanoliposomal composition for immunotherapy comprising:
at least one lipid; and
at least one immunosuppressive drug.

24. The nanoliposomal composition according to claim 23, wherein said at least one lipid comprises cholesterol and at least one phospholipid,
wherein said at least one phospholipid is selected from the group consisting of is phosphatidylserine, phosphatidylethanolamine, and mixtures thereof.

25. The nanoliposomal composition according to claim 24, wherein the Phosphatidylethanolamine is selected from the group consisting of DioleoylPhosphoethanolamine (DOPE), Distearoylphosphoethanolamine (DSPE), phosphatidylcholine, a Distearoylphosphoethanolamine conjugated with polyethylene glycol (PEG) to make Distearoylphosphoethanolamine-polyethylene glycol (DSPE-PEG 2000), and mixtures thereof.

26. The nanoliposomal composition according to claim 25, wherein the DioleoylPhosphoethanolamine (DOPE) is present in an amount from about 10 to about 50% of the total lipid content, and the DSPE-PEG 2000 is present in an amount from about 0.1 to 20% of the total lipid content.

27. The nanoliposomal composition according to claim 25, wherein the phosphatidylcholine is Dipalmitoylphosphatidylcholine (DPPC).

28. The nanoliposomal composition according to claim 27, wherein said Dipalmitoylphosphatidylcholine (DPPC) is present in an amount from about 10 to about 50% of the total lipid content.

29. The nanoliposomal composition according to claim 24, wherein the phosphatidylserine is Dipalmitoylphosphatidylserines (DPPS).

30. The nanoliposomal composition according to claim 29, wherein said Dipalmitoylphosphatidylserines (DPPS) is present in an amount from about 10 to about 50% of the total lipid content.

31. The nanoliposomal composition according to claim 24, wherein said cholesterol is present in an amount from about 1 to about 50%, of the total lipid content.

32. The nanoliposomal composition according to claim 23, wherein the ratio of DioleoylPhosphoethanolamine (DOPE)/Dipalmitoylphosphatidylcholine (DPPC)/dipalmitoylphosphatidylserines (DPPS)/Cholesterol/Distearoylphosphoethanolamine-polyethylene glycol (DSPE-PEG 2000) is about 30:35:10:25:5.

33. The nanoliposomal composition according to claim 23, wherein said at least one immunosuppressive drug is selected from the group consisting of Cyclosporine, Tacrolimus and mixtures thereof.

34. The nanoliposomal composition according to claim 23, wherein said at least one immunosuppressive drug is Cyclosporine A (CsA).

35. The nanoliposomal composition according to claim 34, wherein the total percentage of Cyclospirin A in said nanoliposomal composition does not exceed 5% of the total liposomal composition content.

36. The nanoliposomal composition according to claim 34, wherein the ratio of Cyclospirin A to the total lipid content in said composition ranges from about 1:100 to about 7:100.

37. The nanoliposomal composition according to claim 36, wherein the ratio of Cyclospirin A to the total lipid content in said composition ranges from about 2:70 to about 5:70.

38. The nanoliposomal composition according to claim 23, wherein the nanoliposomes in said nanoliposomal composition have an average diameter of about 100-200 nanometers.

39. The nanoliposomal composition according to claim 38, wherein the nanoliposomes in said nanoliposomal composition have an average diameter of about 100 to 150 nanometers.

40. A method for preparing a liposomal composition for immunotherapy comprising:
preparing a lipid component;
mixing said lipid component with an immunosuppressive drug, and dissolving the admixture in chloroform;
removing the solvent to prepare a thin lipid film;
hydrating the lipid film in a buffer;
separating a lipid film containing vesicles; and
preparing mono-dispersed nanoliposomes.

41. The method for preparing a liposomal composition according to claim 40, wherein the lipid component comprises phospholipids and cholesterol.

42. The method for preparing a liposomal composition according to claim 41, wherein said lipid component comprises at least one phospholipid selected from a group consisting of phosphatidylserine, phosphatidylglycerol, phosphatidylethanolamine, phosphatidylcholine, and mixtures thereof.

43. The method for preparing a liposomal composition according to claim 42, wherein the phosphatidylethanolamine is selected from the group consisting of DioleoylPhosphoethanolamine (DOPE) and Distearoylphosphoethanolamine (DSPE).

44. The method for preparing a liposomal composition according to claim 43, wherein said Distearoylphosphoethanolamine is conjugated with polyethylene glycol (PEG) to make Distearoylphosphoethanolamine-polyethylene glycol compound (DSPE-PEG 2000).

45. The method for preparing a liposomal composition according to claim 42, wherein the phosphatidylserine is Dipalmitoylphosphatidylserine (DPPS).

46. The method for preparing a liposomal composition according to claim 42, wherein the phosphatidylcholine is Dipalmitoylphosphatidylcholine (DPPC).

47. The method for preparing a liposomal composition according to claim 40, wherein said at least one immunosuppressive drug is selected from a group consisting of Cyclosporine, Tacrolimus and mixtures thereof.

48. The method for preparing a liposomal composition according to claim 40, wherein said at least one immunosuppressive drug is Cyclosporine A (CsA).

49. The method for preparing a liposomal composition according to claim 48, wherein the ratio of Cyclosporine A to the total lipid content in said composition ranges from about 1:100 to about 7:100.

50. The method for preparing a liposomal composition according to claim 49, wherein the ratio of Cyclospirin A to the total lipid content in said composition ranges from about 2:70 to about 5:70.

51. The method for preparing a liposomal composition according to claim 40, wherein the ratio of DioleoylPhosphoethanolamine (DOPE)/Dipalmitoylphosphatidylcholine (DPPC)/dipalmitoylphosphatidylserines (DPPS)/Cholesterol/Distearoylphosphoethanolamine-polyethylene glycol (DSPE-PEG 2000) is about 30:35:10:25:5.

52. The method for preparing a liposomal composition according to claim 40, wherein said nanoliposomes have an average diameter of about 90-200 nanometers.

53. The method for preparing a liposomal composition according to claim 52, wherein said nanoliposomes have an average diameter of about 100-150 nanometers.
